(19) United States
(12) Patent Application Publication
(10) Pub. No.: US 2016/0339192 A1
(43) Pub. Date: Nov. 24, 2016

(54) HEAD MOUNTED BREATHING APPARATUS

(71) Applicants: Tsung-En Lee, Taichung (TW); Yen-Hui Huang, Taichung (TW)

(72) Inventors: Tsung-En Lee, Taichung (TW); Yen-Hui Huang, Taichung (TW)

(21) Appl. No.: 15/226,919

(22) Filed: Aug. 3, 2016

(51) Int. Cl. A61M 16/00; A61M 16/06; A61M 16/08

(52) U.S. Cl. CPC A61M 16/0063; A61M 16/0875; A61M 16/0816; A61M 16/0666

(57) ABSTRACT

A head mounted breathing apparatus is provided with an air cylinder filled with pressurized air; a head mount including a band, a fastening member on one side of the band for fastening the air cylinder, and a hook and loop fastener on the band; and a hose assembly including a hose, a connector at one end of the hose for attaching to the air cylinder and having a switch, and a sleeve put on the other end of the hose and having two openings. A person may wear the breathing apparatus when walking, jogging, or running. Further, the person may position the openings to be in close proximity to the nostrils and turn on the switch when he or she has difficulties of breathing. Furthermore, fresh air flows from the air cylinder to the nose via the hose assembly.

### BACKGROUND OF THE INVENTION

1. Field of the Invention

The invention relates to head mounted devices and more particularly to a head mounted breathing apparatus.

2. Description of Related Art

Head mounted devices such as head mounted displays and head mounted lamps are known in the art. However, the present inventors are not aware of a head mounted breathing apparatus disclosed in any patent or non-patent documents.

### SUMMARY OF THE INVENTION

It is therefore one object of the invention to provide a head mounted breathing apparatus comprising an air cylinder filled with pressurized air; a head mount including a band, a fastening member on one side of the band for fastening the air cylinder, and a hook and loop fastener on the band; and a hose assembly including a hose, a connector at one end of the hose for attaching to the air cylinder and having a switch, and a sleeve put on the other end of the hose and having two openings.

The above and other objects, features and advantages of the invention will become apparent from the following detailed description taken with the accompanying drawings.

### BRIEF DESCRIPTION OF THE DRAWINGS

FIG. 1 is a perspective view of a head mounted breathing apparatus according to a first preferred embodiment of the invention;

FIG. 2 is an exploded view of the breathing apparatus;

FIG. 3 is a front view showing the breathing apparatus mounted on the head and in use;

FIG. 4 is a perspective view of the breathing apparatus in a non-operating position;

FIG. 5 is a perspective view of the breathing apparatus in an operating position;

FIG. 6 is an exploded perspective view of a head mounted breathing apparatus according to a second preferred embodiment of the invention;

FIG. 7 is an exploded perspective view of a head mounted breathing apparatus according to a third preferred embodiment of the invention; and

FIG. 8 is a perspective view of a head mounted breathing apparatus according to a fourth preferred embodiment of the invention.

### DETAILED DESCRIPTION OF THE INVENTION

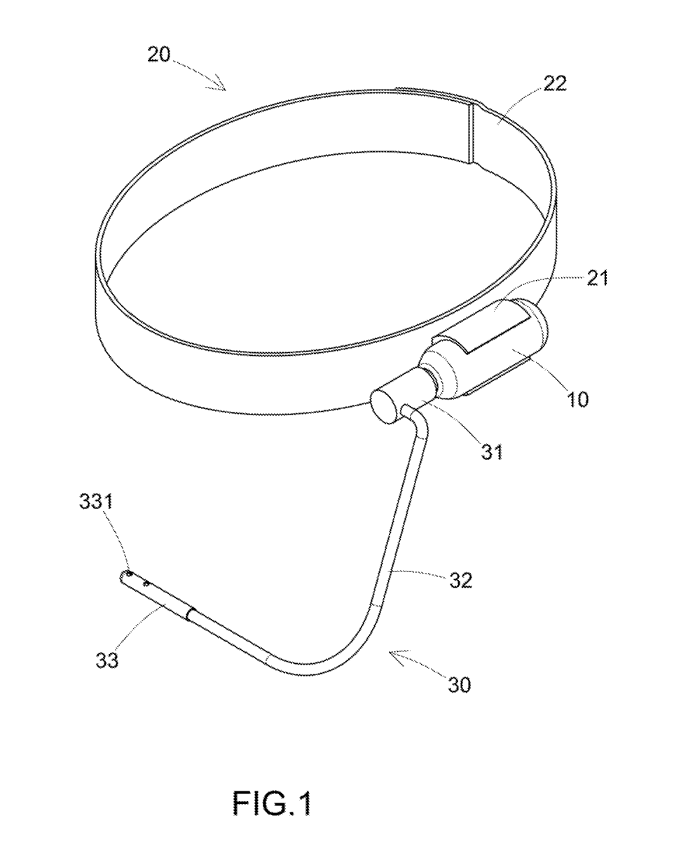

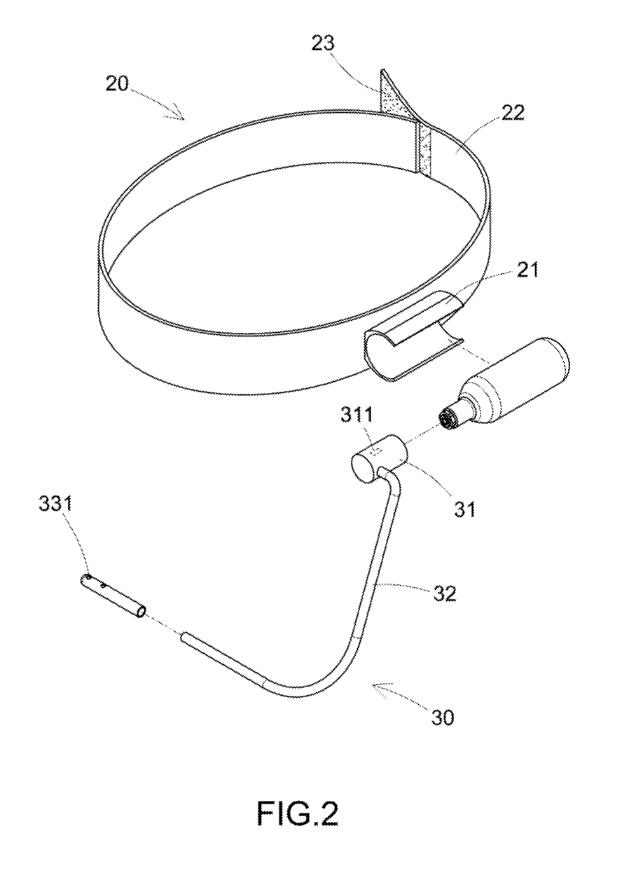

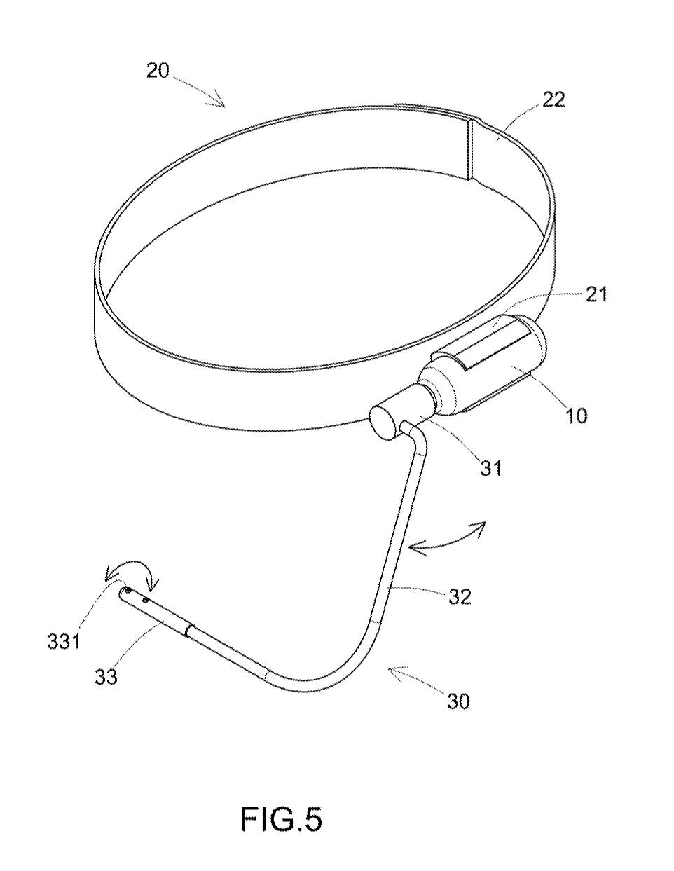

Referring to FIGS. 1 to 5, a head mounted breathing apparatus in accordance with a first preferred embodiment of the invention comprises the following components as discussed in detail below.

An air cylinder 10 is filled with pressurized air. A head mount 20 includes a band 22 made of hard plastic, a fastening member 21 on one side of the band 22 and having a C-shaped section for fastening the air cylinder 10, and a Velcro® fastener 23 on a rear portion of the band 22 for adjusting tightness of the band 22 worn on the head. A hose assembly 30 includes a hose 32, a connector 31 at one end of the hose 32 for attaching to the air cylinder 10 and having a switch 311, and a sleeve 33 put on the other end of the hose 32 and having two openings 331.

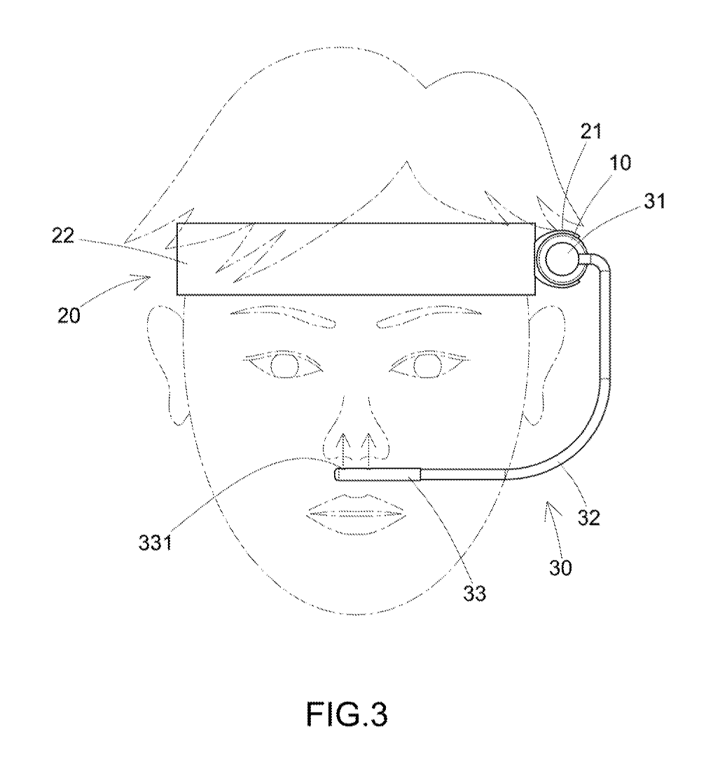

As shown in FIG. 3, a person may wear the breathing apparatus when walking, jogging, or running. Further, the person may position the openings 331 to be in close proximity to the nostrils and turn on the switch 311 when he or she has difficulties of breathing. Thus, fresh air flows from the air cylinder 10 to the nose via the hose assembly 30.

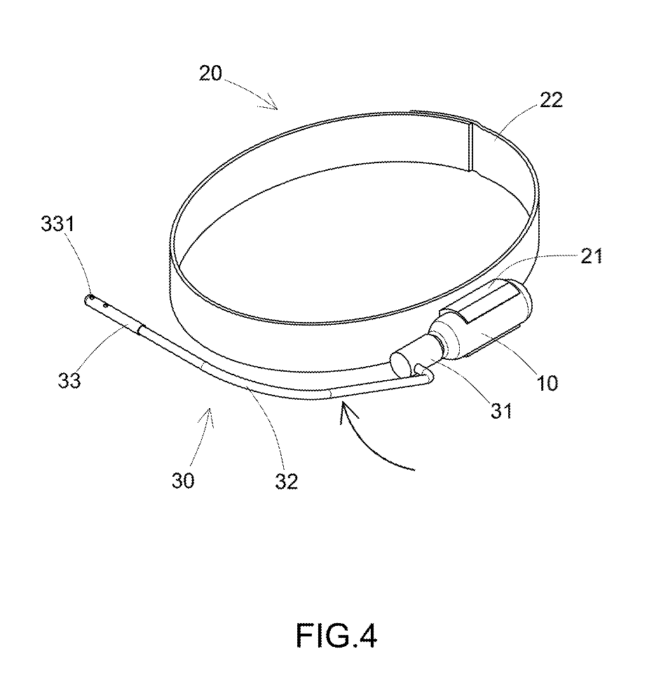

As shown in FIG. 4, in a non-operating position, the switch 311 is turned off and the hose assembly 30 is pushed upward to be in front of the head mount 20.

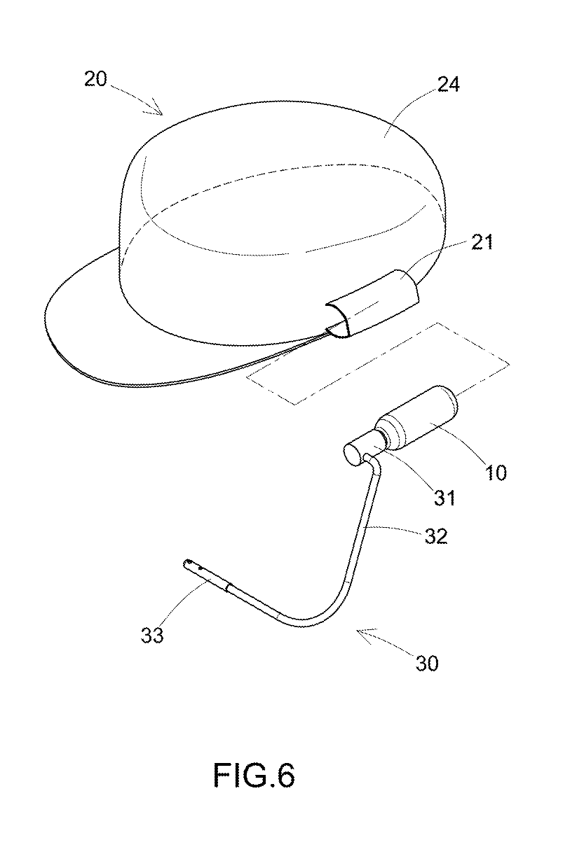

Referring to FIG. 6, a head mounted breathing apparatus in accordance with a second preferred embodiment of the invention is shown. The characteristics of the second preferred embodiment are substantially the same as that of the first preferred embodiment except the following: The band is replaced by a hat 24 and the fastening member 21 is disposed in a direction opposite to that shown in FIG. 1.

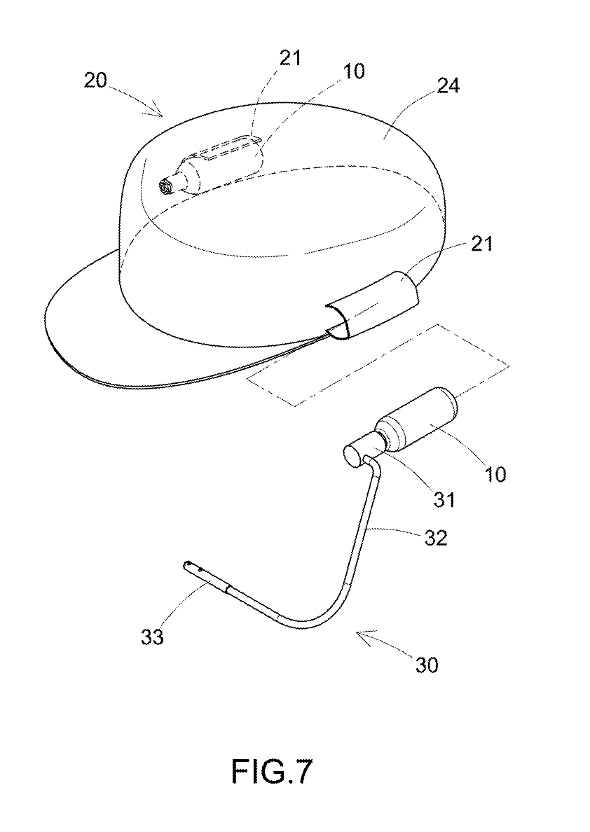

Referring to FIG. 7, a head mounted breathing apparatus in accordance with a third preferred embodiment of the invention is shown. The characteristics of the third preferred embodiment are substantially the same as that of the second preferred embodiment except the following: Two fastening members 21 are provided on two sides of the hat 24 respectively.

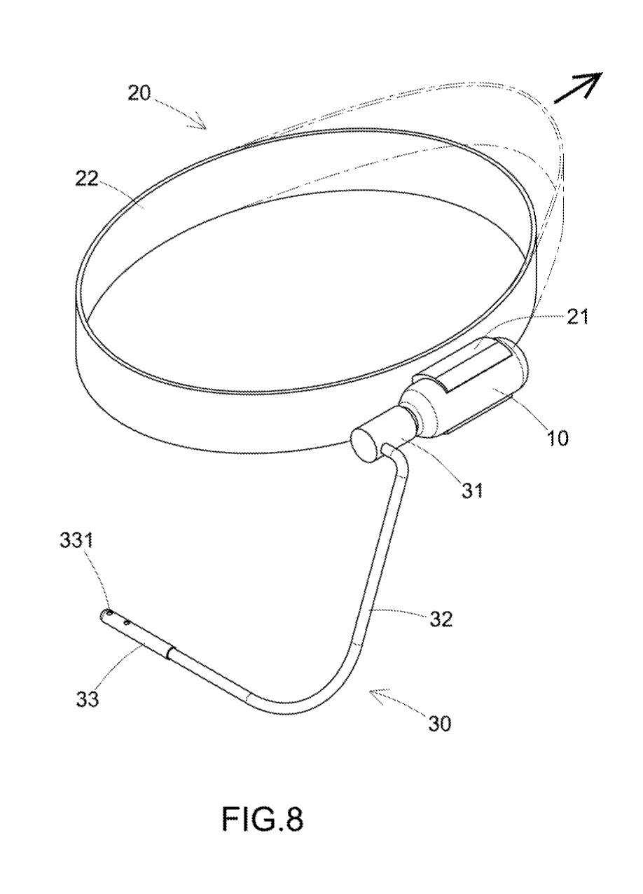

Referring to FIG. 8, a head mounted breathing apparatus in accordance with a fourth preferred embodiment of the invention is shown. The characteristics of the fourth preferred embodiment are substantially the same as that of the first preferred embodiment except the following: The Velcro® fastener 23 is eliminated and the band 22 is made of elastic material so that the band 22 may be expanded (see arrow) or compressed to be held on the head. While the invention has been described in terms of preferred embodiments, those skilled in the art will recognize that the invention can be practiced with modifications within the spirit and scope of the appended claims.

What is claimed is:

1. A breathing apparatus comprising:
an air cylinder filled with pressurized air;
a head mount including a band, a fastening member on one side of the band for fastening the air cylinder, and a hook and loop fastener on the band; and
a hose assembly including a hose, a connector at one end of the hose for attaching to the air cylinder and having a switch, and a sleeve put on the other end of the hose and having two openings.
